SEQUENCE LISTING (17 sequences)

SEQ ID NO: 1 - DNA, 24 bp, Artificial Sequence
  Description of Artificial SequencePCR forward primer OATP8-1F
aggccctgaatgaatattagagaa

SEQ ID NO: 2 - DNA, 24 bp, Artificial Sequence
  Description of Artificial SequencePCR reverse primer OATPF8-1R
taatgtacgcttcaatggaaaaat

SEQ ID NO: 3 - DNA, 24 bp, Artificial Sequence
  Description of Artificial SequencePCR forward primer OATP8-2F
ttactttcttcatctatggaggac

SEQ ID NO: 4 - DNA, 24 bp, Artificial Sequence
  Description of Artificial SequencePCR reverse primer OATP8-2R
aaagctgactctagatgatttgag

SEQ ID NO: 5 - DNA, 24 bp, Artificial Sequence
  Description of Artificial SequencePCR forward primer OATP8-3F
taagatatgcatactggggagaaa

SEQ ID NO: 6 - DNA, 23 bp, Artificial Sequence
  Description of Artificial SequencePCR reverse primer OATP8-3R
ctgcaggatcttaatgggaggtt

SEQ ID NO: 7 - DNA, 24 bp, Artificial Sequence
  Description of Artificial SequencePCR forward primer OATP8-4F
taagatatgcatactggggagaaa

SEQ ID NO: 8 - DNA, 23 bp, Artificial Sequence
  Description of Artificial SequencePCR reverse primer OATP8-4R
ctgcaggatcttaatgggaggtt

SEQ ID NO: 9 - DNA, 24 bp, Artificial Sequence
  Description of Artificial SequencePCR forward primer OATP8-5F
tttgagggaaggtacaatgtcttg

SEQ ID NO: 10 - DNA, 24 bp, Artificial Sequence
  Description of Artificial SequencePCR reverse primer OATP8-5R
tctcaaaaggtaactgcccactta

SEQ ID NO: 11 - DNA, 24 bp, Artificial Sequence
  Description of Artificial SequencePCR forward primer OATP8-6F
tgtaagccaaaccaatggaataat

SEQ ID NO: 12 - DNA, 24 bp, Artificial Sequence
  Description of Artificial SequencePCR reverse primer OATP8-6R
accagaatgcttgatacaatagtg

SEQ ID NO: 13 - DNA, 24 bp, Artificial Sequence
  Description of Artificial SequencePCR forward primer OATP8-7F
aggccctgaatgaatattagagaa

SEQ ID NO: 14 - DNA, 24 bp, Artificial Sequence
  Description of Artificial SequencePCR reverse primer OATP8-7R
taatgtacgcttcaatggaaaaat

SEQ ID NO: 15 - DNA, 500 bp, Homo sapiens
ttcataaatcttgtaatctggatatgtagaaaatataaaaattttaatttctataatttaaaattgttgttcatacaatctagtgtgtggttttatattatttacttgtttcaaatttctctctatgaaaattatttttctaagcaaattataatctctttaggctaggagtttgtctctgtctttcctcctctgtgtccagcattgacctagtcctgtggtcaggaaatagcaggccctgaatgaatattagagaatgattgattgattgatattgagcttgtggcttttcctatttttaaattgtatattgttaaagtaaaataaattatactttttcttttttaacaggtgatcatttcaaaccaagcatcagcaacaattaaaaatattcacttggtatctgtagtttaataatggaccaacatcaacatttgaataaaacagcagagtcagcatcttcagagaaaaagaaaacaagacgctgcaatggattcaag

SEQ ID NO: 16 - DNA, 2646 bp, Homo sapiens
atcagcaacaattaaaatattcacgtggtatctgtagtttaataatggaccaacatcaacatttgaataaaacagcagagtcagcatcttcagagaaaaagaaaacaagacgctgcaatggattcaagatgttcttggcagccctgtcattcagctatattgctaaagcactaggtggaatcattatgaaaatttccatcactcaaatagaaaggagatttgacatatcctcttctcttgctggtttaattgatggaagctttgaaattggaaatttgcttgtgattgtatttgtaagttactttggatctaaactacacagaccgaagttaattggaattggttgtctccttatgggaactggaagtattttgacatctttaccacatttcttcatgggatattataggtattctaaagaaacccatattaatccatcagaaaattcaacatcaagtttatcaacctgtttaattaatcaaaccttatcattcaatggaacatcacctgagatagtagaaaaagattgtgtaaaggaatctgggtcacacatgtggatctatgtcttcatggggaatatgcttcgtggcataggggaaacccccatagtaccattggggatttcatacattgatgattttgcaaaagaaggacattcttccttgtatttaggtagtttgaatgcaataggaatgattggtccagtcattggctttgcactgggatctctgtttgctaaaatgtacgtggatattggatatgtagatctgagcactatcagaataactcctaaggactctcgttgggttggagcttggtggcttggtttccttgtgtctggactattttccattatttcttccataccattttttttcttgccgaaaaatccaaataaaccacaaaaagaaagaaaaatttcactatcattgcatgtgctgaaaacaaatgatgatagaaatcaaacagctaatttgaccaaccaaggaaaaaatgttaccaaaaatgtgactggttttttccagtctttgaaaagcatccttaccaatcccctgtatgttatatttctgcttttgacattgttacaagtaagcagctttattggttcttttacttacgtctttaaatatatggagcaacagtacggtcagtctgcatctcatgctaactttttgttgggaatcataaccattcctacggttgcaactggaatgtttttaggaggatttatcattaaaaaattcaaattgtctttagttggaattgccaaattttcatttcttacttcgatgatatccttcttgtttcaacttctatatttccctctaatctgcgaaagcaaatcagttgccggcctaaccttgacctatgatggaaataattcagtggcatctcatgtagatgtaccactttcttattgcaactcagagtgcaattgtgatgaaagtcagtgggaaccagtctgtgggaacaatggaataacttacctgtcaccttgtctagcaggatgcaaatcctcaagtggtattaaaaagcatacagtgttttataactgtagttgtgtggaagtaactggtctccagaacagaaattactcagcacacttgggtgaatgcccaagagataatacttgtacaaggaaatttttcatctatgttgcaattcaagtcataaactctttgttctctgcaacaggaggtaccacatttatcttgttgactgtgaagattgttcaacctgaattgaaagcacttgcaatgggtttccagtcaatggttataagaacactaggaggaattctagctccaatatattttggggctctgattgataaaacatgtatgaagtggtccaccaacagctgtggagcacaaggagcttgtaggatatataattccgtattttttggaagggtctacttgggcttatctatagctttaagattcccagcacttgttttatatattgttttcatttttgctatgaagaaaaaatttcaaggaaaagataccaaggcatcggacaatgaaagaaaagtaatggatgaagcaaacttagaattcttaaataatggtgaacattttgtaccttctgctggaacagatagtaaaacatgtaatttggacatgcaagacaatgctgctgccaactaacattgcattgattcattaagatgttatttttgaggtgttcctggtctttcactgacaattccaacattctttacttacagtggaccaatggataagtctatgcatctataataaactataaaaaatgggagtacccatggttaggatatagctatgcctttatggttaagattagaatatatgatccataaaatttaaagtgagaggcatggttagtgtgtgatacaataaaaagtaattgtttggtagttgtaactgctaataaaaccagtgactagaatataagggaggtaaaaaggacaagatagattaatagcctaaataaagagaaaagcctgatgcctttaaaaaatgaaacactttggatgtattacttaggccaaaatctggcctggatttatgctataatatatattttcatgttaagttgtatatttttcagaaattataaatattattaatttaaaattcgaaaaaaaaaaaaaaaaaaaaa

SEQ ID NO: 17 - protein, 702 aa, Homo sapiens
MDQHQHLNKTAESASSEKKKTRRCNGFKMFLAALSFSYIAKALGGIIMKISITQIERRFDISSSLAGLIDGSFEIGNLLVIVFVSYFGSKLHRPKLIGIGCLLMGTGSILTSLPHFFMGYYRYSKETHINPSENSTSSLSTCLINQTLSFNGTSPEIVEKDCVKESGSHMWIYVFMGNMLRGIGETPIVPLGISYIDDFAKEGHSSLYLGSLNAIGMIGPVIGFALGSLFAKMYVDIGYVDLSTIRITPKDSRWVGAWWLGFLVSGLFSIISSIPFFFLPKNPNKPQKERKISLSLHVLKTNDDRNQTANLTNQGKNVTKNVTGFFQSLKSILTNPLYVIFLLLTLLQVSSFIGSFTYVFKYMEQQYGQSASHANFLLGIITIPTVATGMFLGGFIIKKFKLSLVGIAKFSFLTSMISFLFQLLYFPLICESKSVAGLTLTYDGNNSVASHVDVPLSYCNSECNCDESQWEPVCGNNGITYLSPCLAGCKSSSGIKKHTVFYNCSCVEVTGLQNRNYSAHLGECPRDNTCTRKFFIYVAIQVINSLFSATGGTTFILLTVKIVQPELKALAMGFQSMVIRTLGGILAPIYFGALIDKTCMKWSTNSCGAQGACRIYNSVFFGRVYLGLSIALRFPALVLYIVFIFAMKKKFQGKDTKASDNERKVMDEANLEFLNNGEHFVPSAGTDSKTCNLDMQDNAAAN

(19) United States
(12) Patent Application Publication
(10) Pub. No.: US 2007/0243528 A1
(43) Pub. Date: Oct. 18, 2007

(54) Methods for detecting polymorphisms using arms or rflp

(75) Inventors: Helen Jean Ambrose, Macclesfield (GB); Adam Jeston Dudley, Wilmington, DE (US)

Correspondence Address: FISH & RICHARDSON P.C., P.O BOX 1022, MINNEAPOLIS, MN, 55440-1022, US

(73) Assignee: ASTRAZENECA AB

(21) Appl. No.: 10/517,688

(22) PCT Filed: Jun. 10, 2003
(86) PCT No.: PCT/GB03/02524

§ 371 Date: Dec. 10, 2004

Related U.S. Application Data
(60) Provisional application No. 60/388,812, filed on Jun. 14, 2002.

(30) Foreign Application Priority Data
Jun. 13, 2002 (GB) 0213579.6

(51) Int. Cl. C12Q 1/68; C12P 19/34

(52) U.S. Cl. 435/6; 435/91.2

(57) ABSTRACT

The invention provides a method for the detection of a polymorphism in OATP8 in a human. The method for detection of a nucleic acid polymorphism is selected from amplification refractory mutation system and restriction fragment length polymorphism. The invention also provides use of the method to assess the pharmacogenetics of a drug transportable by OAT8.

This invention relates to polymorphisms in the human OATP8 gene and corresponding novel allelic polypeptides encoded thereby. The invention also relates to methods and materials for analysing allelic variation in the OATP8 gene, and to the use of OATP8 polymorphism in treatment of diseases with OATP8 transportable drugs.

Membrane transporters are important for the absorption of oral medications across the gastrointestinal tract, uptake in to target tissues such as the liver or brain, and excretion into the bile and urine. Changes in the activities of transporters may therefore have a significant effect on the bioavailability of clinically important drugs (Hiroyuki Kusuhara et al. Journal of Controlled Release 78 (2002) 43-54).

It has been reported in the literature that polymorphisms in proteins involved in drug transport can alter the function of the protein. For example, the multidrug-resistance (MDR-1) gene contains a polymorphism in exon 26 (C3435T) which has been correlated with expression levels and function of MDR-1. Individuals homozygous for this polymorphisms have significantly lower duodenal MDR-1 expression and high digoxin plasma levels, suggesting this polymorphism affects the absorption and tissue concentrations of substrates of MDR-1 (S Hoffmeyer et al. Proceedings National Academy Science (2000) 97, 3473-3478).

The human sodium independent organic anion transporting polypeptide (OATP) 8 gene is a member of the OATP supergene family involved in multifunctional transport of organic anions (Rommel G et al. Journal of Biological Chemistry 276: 35669-35675 (2001); Ikumi Tamai et al. Biochemical and Biophysical Research Communications 273, 251-260 (2000)). There is an alternative nomenclature for this family as SLC21A (solute carriers) and OATP8 relates to SLC21A8. OATP8 has been cloned (Jörg König et al. Journal of Biological Chemistry 275: 23161-23168 (2000)), and a cDNA sequence encoding OATP8 has been submitted to the EMBL database under accession number AJ251506.

OATP8 has a 79% identity at the amino acid level with its gene family member human OATPC (SLC21A6) and is found on the same gene cluster on chromosome 12. OATPC has broad tissue specificity and is considered to play a major role in hepatic uptake of organic anions including xenobiotics (Gerd A et al. Gastroenterology 2001 120: 525-533). OATPC has been shown to be involved in the transport of drugs including benzylpenicillin and those involved in lipid lowering e.g. statins (Daisuke Nakai et al. J Pharmacol Exp Ther 2001 297: 861-867). Statins have been referred to as a first-line therapy for patients with atherosclerotic vascular diseases (Bonnie Hsiang et al. J. Biol Chem 274, 37161-37168 (1999)). OATPC is localized at the basolateral hepatocyte membrane. OATP8 has also been localised to the basolateral hepatocyte membrane. Due to its sequence homology, it is likely that OATP8 may transport similar substrates as OATPC. Indeed, OATP8 has been shown to transport the same organic anions as OATPC, such as sulfobromophthalein and 17β-glucuronosyl estradiol. OATP8 also appears to specifically transport some cardiac glycosides including digoxin. As OATP8 is a liver-specific transporter, the protein coded for by the OATP8 gene may be useful in many liver-specific drug delivery systems.

A screen for polymorphisms in OATS8 in 48 Japanese individuals has been published (Artoshi Iida et al. Journal of Human Genetics (2001) 46:668-683) and identifies 80 polymorhpisms in OATP8, of which 4 lie in exons. These are Ser112Ala (Exon 3), Leu424Leu (Exon 9), Ala519Ala (Exon 11) and Gly611Gly (Exon 13). The allele frequencies for the individual SNPs are not reported, just the percentage of types of SNPs found.

DNA polymorphisms are variations in DNA sequence between one individual and another. DNA polymorphisms may lead to variations in amino acid sequence and consequently to altered protein structure and functional activity. Polymorphisms may also affect mRNA synthesis, maturation, transportation and stability. Polymorphisms which do not result in amino acid changes (silent polymorphisms) or which do not alter any known consensus sequences may nevertheless have a biological effect, for example by altering mRNA folding or stability.

Knowledge of polymorphisms may be used to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The genomic DNA sequence (AC011604) immediately upstream of the OATP8 protein coding sequence is set out as SEQ ID NO: 15, with the first nucleotide of the OATP8 coding region accorded position 417. The position of the polymorphisms in the genomic DNA sequence are defined with reference to SEQ ID NO: 15 unless stated otherwise or apparent from the context.

A cDNA sequence encoding OATP8 (AJ251506) is set out as SEQ ID NO: 16, with the first nucleotide of the OATP8 coding region accorded position 45. All positions of polymorphisms in the human OATP8 gene transcribed into mRNA (and thence cDNA) herein refer to the positions in SEQ ID NO: 16 unless stated otherwise or apparent from the context.

All positions herein of polymorphisms in the OATP8 polypeptide are defined with reference to SEQ ID NO: 17 unless stated otherwise or apparent from the context.

The present invention is based on the discovery of 10 polymorphisms in the human OATP8 gene. The polymorphisms of the present invention may alter the transport of pharmaceutical agents.

According to one aspect of the present invention there is provided a method for the detection of a polymorphism in OATP8 in a human, which method comprises determining the sequence of the human at any one of the following positions: positions 743, 811, 2021 and 2380 of SEQ ID NO: 16; positions 233 and 256 of SEQ ID NO: 17.

According to a further aspect of the present invention there is provided a method for the detection of a polymorphism in OATP8 in a human, which method comprises (ii) determining the sequence of the human, wherein the human is a Caucasian human, at any one of the following positions: positions 389, 410 and 389-392 of SEQ ID NO: 15; positions 378, 1877 and 2501-2505 of SEQ ID NO: 16; position 112 of SEQ ID NO: 17.

The term “human” includes both a human having or suspected of having an OATP8 mediated response to a drug and an asymptomatic human who may be tested for predisposition or susceptibility to such a response. At each position the human may be homozygous for an allele or the human may be a heterozygote.

The term “detection of a polymorphism” refers to determination of the genetic status of an individual at a polymorphic position (in which the individual may be homozygous or heterozygous at each position).

The term “OATP8 mediated response” means any disease in which changing the level of an OATP8 mediated response or changing the biological activity of OATP8 would be of therapeutic benefit.

The term “polymorphism” includes nucleotide substitution, nucleotide insertion and nucleotide deletion, which in the case of insertion and deletion includes insertion or deletion of one or more nucleotides at a position of a gene and variable numbers of a repeated DNA sequence.

In one embodiment of the invention preferably the polymorphism is further defined as:
polymorphism at position 389 is presence of A and/or T;
polymorphism at position 410 is presence of T and/or A;
polymorphism at position 389-392 is presence of ATAT and/or TAGA;
polymorphism at position 743 is presence of A and/or G;
polymorphism at position 811 is presence of G and/or C;
polymorphism at position 2021 is presence of G and/or A;
polymorphism at position 2380 is presence of A and/or T;
polymorphism at position 378 is presence of G and/or T;
polymorphism at position 1877 is presence of A and/or G;
polymorphism at position 2501-2505 is presence of AAAAA and/or AAAAAA;
polymorphism at position 233 is presence of Ile and/or Met;
polymorphism at position 256 is presence of Gly and/or Ala; and
polymorphism at position 112 is presence of Ser and/or Ala.

The polymorphism at position 2501-2505 of SEQ ID NO: 16 is the result of an insertion event defined as insertion of A at position 2501 of SEQ ID NO: 16. Because this insertion occurs in a run of A's, the precise position is not definitive but merely changes (A)₅ to (A)₆ in this run of A's. This results in an overall extra one base and it will be appreciated by the skilled reader that this will have an effect on the numbering of positions downstream of this. For example, position 2501 of SEQ ID NO: 16 becomes position 2502 after the insertion event. It will also be appreciated by the skilled person that it may not be necessary to sequence the exact 2501-2505 bases or the insertion at this position to distinguish between the two alleles. For example, position 2506 is either T or A when comparing the sequence of the two alleles and this is within the scope of the invention.

The polymorphism at position 389 of SEQ ID NO: 15 is the result of a deletion event defined as deletion of bases 389-406 of SEQ ID NO: 15. This results in 18 bases less overall and it will be appreciated by the skilled person that this will have an effect on the numbering of the positions downstream of this. For example, position 407 of SEQ ID NO: 15 becomes position 389 after the deletion event. It will also be appreciated by the skilled person that it may not be necessary to sequence the entire 389406 bases or the deletion at this position to distinguish between the two alleles. For example, position 389 is either an A or a T when comparing the sequence of the two alleles.

The polymorphism at position 410 of SEQ ID NO: 15 is the result of a deletion event defined as deletion of bases 410413 of SEQ ID NO: 15. This results in 4 bases less overall and it will be appreciated by the skilled person that this will have an effect on the numbering of the positions downstream of this. For example, position 414 of SEQ ID NO: 15 becomes position 410 after the deletion event. It will also be appreciated by the skilled person that it may not be necessary to sequence the entire 410-413 bases or the deletion at this position to distinguish between the two alleles. For example, position 410 is either a T or an A when comparing the sequence of the two alleles.

The polymorphism at position 389-392 of SEQ ID NO: 15 is the result of a double deletion event defined as deletion of bases 389406 and 410-413 of SEQ ID NO: 15. This results in 22 bases less overall and it will be appreciated by the skilled person that this will have an effect on the numbering of the positions downstream of this. For example, position 414 of SEQ ID NO: 15 becomes position 392 after the double deletion event. It will also be appreciated by the skilled person that it may not be necessary to sequence the entire 389406 and 410413 bases or the deletion at these positions to distinguish between the two alleles. For example, positions 389-392 are either ATAT or TAGA when comparing the sequence of the two alleles.

Preferred methods for detection of nucleic acid polymorphism are amplification refractory mutation system and restriction fragment length polymorphism.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| FRET | Fluorescence resonance energy transfer |
| LCR | Ligase chain reaction |
| MASDA | Multiple allele specific diagnostic assay |
| NASBA | Nucleic acid sequence based amplification |
| OATP | Na+-independent organic anion transporting polypeptide |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |
| UTR | Untranslated region |

Table 1—Mutation Detection Techniques
General: DNA sequencing, Sequencing by hybridisation
Scanning: PIT*, SSCP, DGGE, TGGE, Cleavase, Heteroduplex analysis, CMC, Enzymatic mismatch cleavage
Note: not useful for detection of promoter polymorphisms.

Hybridisation Based
Solid phase hybridisation: Dot blots, MASDA, Reverse dot blots, Oligonucleotide arrays (DNA Chips)

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)
Extension Based: ARMS™, ALEX™—European Patent No. BP 332435 B1 (Zeneca Limited), COPS—Gibbs et al (1989), Nucleic Acids Research, 17,2347.
Incorporation Based: Mini-sequencing, APEX
Restriction Enzyme Based: FELP, Restriction site generating PCR
Ligation Based: OLA
Other: Invader assay

Table 2—Signal Generation or Detection Systems
Fluorescence: FRET, Fluorescence quenching, Fluorescence polarisation—United Kingdom Patent No. 2228998 (Zeneca limited)
Other: Chemiluminescence, Electrochemiluminescence, Raman, Radioactivity, Colorimetric, Hybridisation protection assay, Mass spectrometry

Table 3—Further Amplification Methods
SSR, NASBA, LCR, SDA, b-DNA

Table 4—Protein Variation Detection Methods
Immunoassay
Immunohistology
Peptide sequencing

Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom. Histological techniques are known in the art e.g. Theory and Practice of Histological Techniques, 4^{th} Edition, edited by J D Bancroft and A Stevens, Churchill Livingstone, 1996. Protein sequencing is described in Laboratory techniques Biochemistry and Molecular Biology, Volume 9, Sequencing of Proteins and Polypeptides, G Allen, 2^{nd} revised edition, Elsevier, 1989.

Preferred mutation detection techniques include ARMS™, ALEX™, COPS, Taqman, Molecular Beacons, RFLP, restriction site based PCR and FRET techniques, polyacrylamide gel electrophoresis and capillary electrophoresis.

Particularly preferred methods include ARMS™ and RFLP based methods. ARMS™ is an especially preferred method.

In a further aspect, the methods of the invention are used to assess the pharmacogenetics of a drug transportable by OATP8.

Assays, for example reporter-based assays, may be devised to detect whether one or more of the above polymorphisms affect transcription levels and/or message stability.

Individuals who carry particular allelic variants of the OATP8 gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The methods of the invention may be useful both to predict the clinical response to such agents and to determine therapeutic dose.

In a further aspect, the methods of the invention, are used to assess the predisposition and/or susceptibility of an individual to diseases mediated by OATP8. The present invention may be used to recognise individuals who are particularly at risk from developing such diseases.

In a further aspect, the methods of the invention are used in the development of new drug therapies which selectively target one or more allelic variants of the OATP8 gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further aspect of the invention the presence or absence of variant nucleotides is detected by reference to the loss or gain of, optionally engineered, sites recognised by restriction enzymes.

According to another aspect of the present invention there is provided a human OATP8 gene or its complementary strand comprising a variant allelic polymorphism at one or more of positions defined herein or a fragment thereof of at least 20 bases comprising at least one novel polymorphism

Fragments are at least 17 bases, more preferably at least 20 bases, more preferably at least 30 bases.

According to another aspect of the present invention there is provided a polynucleotide comprising at least 20 contiguous bases of the human OATP8 gene and comprising an allelic variant selected from any of the following:

| Region | variant | Position | |
|---|---|---|---|
| Exon 6 | G | 743 | (SEQ ID NO: 16) |
| Exon 7 | C | 811 | (SEQ ID NO: 16) |
| Exon 14 | A | 2021 | (SEQ ID NO: 16) |
| 3′ UTR | T | 2380 | (SEQ ID NO: 16) |

According to a further aspect of the present invention there is provided a polynucleotide comprising at least 20 contiguous bases of the human OATP8 gene, wherein the human is a Caucasian human, and comprising an allelic variant selected from any of the following:

| Region | variant | Position | |
|---|---|---|---|
| 5′UTR | T | 389 | (SEQ ID NO: 15) |
| 5′UTR | A | 410 | (SEQ ID NO: 15) |
| 5′UTR | TAGA | 389-392 | (SEQ ID NO: 15) |
| Exon 3 | T | 378 | (SEQ ID NO: 16) |
| Exon 13 | G | 1877 | (SEQ ID NO: 16) |
| 3′ UTR | AAAAAA | 2501-2505 | (SEQ ID NO: 16) |

According to another aspect of the present invention there is provided a human OATP8 gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting an OATP8 gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting an OATP8 gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting an OATP8 gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the polymorphisms of this invention may be used as genetic markers in linkage studies. This particularly applies to the polymorphisms of relatively high frequency. The OATP8 gene is on chromosome 12. Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that polymorphisms with low frequency alleles are particularly useful as markers of low frequency haplotypes. As particular mutations or polymorphisms associated with certain clinical features, such as adverse or abnormal events, are likely to be of low frequency within the population, low frequency polymorphisms may be particularly useful in identifying these mutations (for examples see: De Stefano V et al. Ann Hunt Genet (1998) 62:481-90;and Keightley A M et al. Blood (1999) 93:4277-83).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis or any other bioinformatic analysis. The reader is referred to Bioinformatics, A practical guide to the analysis of genes and proteins, Edited by A D Baxevanis & B F F Ouellette, John Wiley & Sons, 1998. Any computer readable medium may be used, for example, compact disk, tape, floppy disk, hard drive or computer chips.

The polynucleotide sequences of the invention, or parts thereof, particularly those relating to and identifying the polymorphisms identified herein represent a valuable information source, for example, to characterise individuals in terms of haplotype and other sub-groupings, such as investigation of susceptibility to treatment with particular drugs. These approaches are most easily facilitated by storing the sequence information in a computer readable medium and then using the information in standard bioinformatics programs or to search sequence databases using state of the art searching tools such as “GCC”. Thus, the polynucleotide sequences of the invention are particularly useful as components in databases useful for sequence identity and other search analyses. As used herein, storage of the sequence information in a computer readable medium and use in sequence databases in relation to polynucleotide or polynucleotide sequence of the invention covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data.

The invention provides a computer readable medium having stored thereon one or more polynucleotide sequences of the invention. For example, a computer readable medium is provided comprising and having stored thereon a member selected from the group consisting of: a polynucleotide comprising the sequence of a polynucleotide of the invention, a polynucleotide consisting of a polynucleotide of the invention, a polynucleotide which comprises part of a polynucleotide of the invention, which part includes at least one of the polymorphisms of the invention, a set of polynucleotide sequences wherein the set includes at least one polynucleotide sequence of the invention, a data set comprising or consisting of a polynucleotide sequence of the invention or a part thereof comprising at least one of the polymorphisms identified herein.

A computer based method is also provided for performing sequence identification, said method comprising the steps of providing a polynucleotide sequence comprising a polymorphism of the invention in a computer readable medium; and comparing said polymorphism containing polynucleotide sequence to at least one other polynucleotide or polypeptide sequence to identify identity (homology), i.e. screen for the presence of a polymorphism.

According to another aspect of the present invention there is provided a method of treating a human in need of treatment with a drug transportable by OATP8 in which the method comprises:
i) detection of a polymorphism in OATP8 in a human, which method comprises determining the sequence of the human at one of the following positions: positions 743, 811, 2021, 2380 of SEQ ID NO: 16; positions 233 and 256 of SEQ ID NO: 17; and
ii) administering an effective amount of the drug.

In a further aspect of the present invention there is provided a method of treating a Caucasian human in need of treatment with a drug transportable by OATP8 in which the method comprises:
i) detection of a polymorphism in OATP8 in a human, which method comprises determining the sequence of the human, wherein the human is a Caucasian human, at one of the following positions: positions 389, 410 and 389-392 of SEQ ID NO: 15; positions 378, 1877 and 2501-2505 of SEQ ID NO: 16; position 112 of SEQ ID NO: 17; and
ii) administering an effective amount of the drug.

Preferably determination of the status of the human is clinically useful. Examples of clinical usefulness include deciding which statin drug or drugs to administer and/or in deciding on the effective amount of the statin drug or drugs. Statins already approved for use in humans include atorvastatin, cerivastatin, fluvastatin, pravastatin and simvastatin. Statins under development include rosuvastatin. The reader is referred to the following references for further information: Drugs and Therapy Perspectives (12^{th} May 1997), 9: 1-6; Chong (1997) Pharmacotherapy 17: 1157-1177;Kellick (1997) Formulary 32: 352; Kathawala (1991) Medicinal Research Reviews, 11: 121-146; Jahng (1995) Drugs of the Future 20: 387404, and Current Opinion in Lipidology, (1997), 8, 362-368; Olsson A G, Pears J, McKellar J, Mizan J & Raza A (2001) American Journal of Cardiology 88(5): 504-8. The term “drug transportable by OATP8” means that transport by OATP8 in humans is an important part of a drug exerting its pharmceutical effect in man. For example, some statins have to be transported to the liver by OATPC, which is highly homologous to OATP8, to exert their lipid lowering effects. Accordingly, OATP8 is expected to be involved in statin transport.

According to another aspect of the present invention there is provided use of a drug transportable by OATP8 in preparation of a medicament for treating a disease in a hum determined as having a polymorphism defined herein.

According to another aspect of the present invention there is provided a pharmaceutical pack comprising OATP8 transportable drug and instructions for administration of the drug to humans tested for a polymorphism described therein, preferably at one or more of the positions defined herein.

Three of the polymorphisms of the present invention result in variation in the amino acid sequence of the translated protein. Polymorphism at position 743 as defined in SEQ ID NO: 16 results in an amino acid change from isoleucine to methionine at corresponding position 233 of the translated protein (Ile233Met) as defined in SEQ ID NO: 17. Polymorphism at position 811 as defined in SEQ ID NO: 16 results in an amino acid change from glycine to alanine at corresponding position 256 of the translated protein (Gly256Ala) as defined in SEQ ID NO: 17. Polymorphism at position 378 as defined in SEQ ED NO: 16 results in an amino acid change from serine to alanine at corresponding position 112 of the translated protein (Ser112Ala) as defined in SEQ ID NO: 17.

Thus according to another aspect of the present invention there is provided an allelic variant of human OATP8 polypeptide comprising:
a methionine at position 233 of SEQ ID NO: 17;
an alanine at position 256 of SEQ ID NO: 17;
an alanine at position 112 of SEQ ID NO: 17;
or a fragment thereof comprising at least 10 amino acids provided that the fragment comprises the allelic variant at position 233, 256 or 112 of SEQ ID NO: 17.

Fragments of OATP8 polypeptide are at least 10 amino acids, more preferably at least 15 amino acids, more preferably at least 20 amino acids. The polypeptides of the invention do not encompass naturally occurring polypeptides as they occur in nature, for example, the polypeptide is at least partially purified from at least one component with which it occurs naturally. Preferably the polypeptide is at least 30% pure, more preferably at least 60% pure, more preferably at least 90% pure, more preferably at least 95% pure, and more preferably at least 99% pure.

According to another aspect of the present invention there is provided an antibody specific for an allelic variant of human OATP8 polypeptide as described herein comprising:
a methionine at position 233 of SEQ ID NO: 17;
an alanine at position 256 of SEQ ID NO: 17;
an alanine at position 112 of SEQ ID NO: 17;
or a fragment thereof comprising at least 10 amino acids provided that the fragment comprises the allelic variant at position 233, 256 or 112 of SEQ ID NO: 17.

Antibodies can be prepared using any suitable method. For example, purified polypeptide may be utilized to prepare specific antibodies. The term “antibodies” is meant to include polyclonal antibodies, monoclonal antibodies, and the various types of antibody constructs such as for example F(ab′)₂, Fab and single chain Fv. Antibodies are defined to be specifically binding if they bind the allelic variant of OATP8 with a Kₐ of greater than or equal to about 10⁷ M⁻¹ Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit comprising an antibody of the invention.

The invention will now be illustrated but not limited by reference to the following examples. All temperatures are in degrees Celsius.

In the examples below, unless otherwise stated, the following methodology and materials have been applied.

AMPLITAQ™ available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or in “Current Protocols in Molecular Biology Volumes 1-3,edited by F M Asubel, R Brent and RE Kingston; published by John Wiley, 1998.

Electropherograms were obtained in a standard manner data was collected by ABI377 data collection software and the wave form generated by ABI Prism sequencing analysis (2.1.2).

### EXAMPLES

### Example 1

Identification of Polymorphisms

1. Methods

The cDNA sequence of OATP8 (AJ251506) was used to identify the genomic position of the gene and obtain genomic DNA sequence. The genomic DNA sequence was used to design PCR primers to amplify over the exons and intron/exon boundaries of the full length of the OATP gene and some regions upstream of exon 1 and downstream of the final exon. Twenty-nine individual Caucasian genomic DNA samples were used as templates for PCR amplification. The products were then sequenced by dye-primer sequencing. The alignment of sequence traces enabled the identification of polymorphisms. The frequency of the polymorphisms was confirmed by RFLP and primer extension analysis by HPLC (WAVE) method using genomic DNA from 29 individuals and by sequencing. Deletion events were identified by sequencing and allele frequencies determined by amplification across the deletion.

PCR Products

| Polymorphism | PCR forward oligo | PCR reverse oligo |
|---|---|---|
| Exon 6 | (SEQ ID NO: 1) | (SEQ ID NO: 2) |
| Exon 7 | (SEQ ID NO: 3) | (SEQ ID NO: 4) |
| Exon 14 | (SEQ ID NO: 5) | (SEQ ID NO: 6) |
| 3′UTR | (SEQ ID NO: 7) | (SEQ ID NO: 8) |
| Exon 3 | (SEQ ID NO: 9) | (SEQ ID NO: 10) |
| Exon 13 | (SEQ ID NO: 11) | (SEQ ED NO: 12) |
| 3′UTR (position 2501- | (SEQ ID NO: 7) | (SEQ ID NO: 8) |
| 2505 of SEQ ID NO: 16) | | |
| 5′UTR (position 389 of | (SEQ ID NO: 13) | (SEQ ID NO: 14) |
| SEQ ID NO: 15) | | |
| 5′UTR (position 410 of | (SEQ ID NO: 13) | (SEQ ID NO: 14) |
| SEQ ID NO: 15) | | |
| 5′UTR (position 389-392 | (SEQ ID NO: 13) | (SEQ ID NO: 14) |
| of SEQ ID NO: 15) | | |

PCR conditions:
1x GeneAmp® PCR buffer II (Roche Molecular Systems Inc.)
100 μM of each of dATP, dCTP, dGTP, dTTP
1.5 mM MgC₂
0.5 μM of forward and reverse primers
1Unit AmpliTaq Gold® per 25 μl PCR reaction (Roche Molecular Systems Inc.)
   PCR programme:
95° C. 10 min; (94° C. 30 sec, 60° C. 30 sec, 72° C. 90 sec) for 35 cycles; 72° C. 10 min.
   2. OATP8 Polymorphisms

Sequencing of DNA from 29 individuals identified the following 10 polymorphisms in the OATP8 DNA sequence:

| | | | Resultant | Protein | |
|---|---|---|---|---|---|
| | | | codon | sequence | Frequency |
| Position | Region | Variation | change | SEQ ID NO 17 | variant allele |
| 389 (SEQ | 5′UTR | A/T | N/a | N/a | 0.24 |
| ID NO: 15) | | | | | |
| 410 (SEQ | 5′UTR | T/A | N/a | N/a | 0.24 |
| ID NO: 15) | | | | | |
| 389-392 | 5′UTR | ATAT/ | N/a | N/a | 0.24 |
| (SEQ ID NO: 15) | | TAGA | | | |
| 743 (SEQ | Exon 6 | A/G | ATA- | Ile233Met | 0.08G |
| ID NO: 16) | | | ATG | | |
| 811 (SEQ | Exon 7 | G/C | GGA- | Gly256Ala | 0.16C |
| ID NO: 16) | | | GCA | | |
| 2021 (SEQ | Exon 14 | G/A | TCG- | Ser659Ser | 0.06A |
| ID NO: 16) | | | TCA | | |
| 2380 (SEQ | 3′UTR | A/T | N/a | N/a | 0.92T |
| ID NO: 16) | | | | | |
| 378 (SEQ | Exon 3 | G/T | TCT- | Ser112Ala | 0.09T |
| ID NO: 16) | | | GCT | | |
| 1877 (SEQ | Exon 13 | A/G | GGA- | Gly611Gly | 0.08G |
| ID NO: 16) | | | GGG | | |
| 2501-2505 | 3′UTR | AAAAA/ | N/a | N/a | 0.25AAAAAA |
| (SEQ ID NO: 16) | | AAAAAA | | | |

1. A method for the detection of a polymorphism in OATP8 in a human which method comprises:
(i) determining the sequence of the human at any one of the following positions:
positions 743, 811, 2021 and 2380 of SEQ ID NO: 16;
positions 233 and 256 of SEQ ID NO: 17; or
(ii) determining the sequence of the human, wherein the human is a Caucasian human, at any one of the following positions:
positions 389, 410 and 389-392 of SED ID NO: 15;
positions 378, 1877 and 2501-2505 of SEQ ID NO: 16;
position 112 of SEQ ID NO: 17.

2. A method according to claim 1 wherein the polymorphism is further defined as:
polymorphism at position 389 is presence of A and/or T;
polymorphism at position 410 is presence of T and/or A;
polymorphism at position 389-392 is presence of ATAT and/or TAGA;
polymorphism at position 743 is presence of A and/or G;
polymorphism at position 811 is presence of G and/or C;
polymorphism at position 2021 is presence of G and/or A;
polymorphism at position 2380 is presence of A and/or T;
polymorphism at position 378 is presence of G and/or T;
polymorphism at position 1877 is presence of A and/or G;
polymorphism at position 2501-2505 is presence of AAAAA and/or AAAAAA;
polymorphism at position 233 is presence of Ile and/or Met;
polymorphism at position 256 is presence of Gly and/or Ala; and
polymorphism at position 112 is presence of Ser and/or Ala.

3. A method according to claim 1 wherein the method for detection of a nucleic acid polymorphism is selected from amplification refractory mutation system and restriction fragment length polymorphism.

4. Use of a method defined in claim 1 to assess the pharmacogenetics of a drug transportable by OATP8.

5. A polynucleotide comprising at least 20 contiguous bases of the human OATP8 gene and comprising an allelic variant selected from any of the following:

| Region | variant | Position | |
|---|---|---|---|
| Exon 6 | G | 743 | (SEQ ID NO: 16) |
| Exon 7 | C | 811 | (SEQ ID NO: 16) |
| Exon 14 | A | 2021 | (SEQ ID NO: 16) |
| 3′ UTR | T | 2380 | (SEQ ID NO: 16) |

6. An allele specific primer capable of detecting an OATP8 gene polymorphism at one of the following positions: positions 389, 410 and 389-392 of SEQ ID NO: 15; positions 743, 811, 2021, 2380, 378, 1877 and 2501-2505 of SEQ ID NO: 16.

7. An allele specific oligonucleotide probe capable of-detecting a OATP8 gene polymorphism at one of the following positions: positions 389, 410 and 289-392 of SEQ ID NO: 15; positions 743, 811, 2021, 2380, 378, 1877 and 2501-2505 of SEQ ID NO: 16.

8. A diagnostic kit comprising the allele-specific primer of claim 6.

9. A method of treating a human in need of treatment with a drug transportable by OATP8 in which the method comprises detection of a polymorphism in OATP8 in a human, which method comprises:
(i) determining the sequence of the human at one of the following positions:
positions 743, 811, 2021, 2380 of SEQ ID NO: 16;
positions 233 and 256 of SEQ ID NO: 17; or
determining the sequence of the human, wherein the human is a Caucasian human, at one of the following positions:
positions 389, 410 and 389-392 of SEQ ID NO: 15;
positions 378, 1877 and 2501-2505 of SEQ ID NO: 16;
position 112 of SEQ ID NO: 17; and
ii) administering an effective amount of the drug.

10. Use of a drug transportable by OATP8 in preparation of a medicament for treating a disease in a human determined as having a polymorphism at one of the following positions:
positions 389, 410 and 389-392 of SEQ ID NO: 15;
positions 743, 811, 2021, 2380, 378, 1877 and 2501-2505 of SEQ ID NO: 16;
positions 233, 256 and 112 of SEQ ID NO: 17.

11. An allelic variant of human OATP8 polypeptide comprising:
a methionine at position 233 of SEQ ID NO: 17;
an alanine at position 256 of SEQ ID NO: 17;
an alanine at position 112 of SEQ ID NO: 17;
or a fragment thereof comprising at least 10 amino acids provided that the fragment comprises the allelic variant at position 233, 256 or 112 of SEQ ID NO: 17.

12. An antibody specific for an allelic variant of human OATP8 polypeptide as described herein having:
a methionine at position 233 of SEQ ID NO: 17;
an alanine at position 256 of SEQ ID NO: 17;
an alanine at position 112 of SEQ ID NO: 17;
or a fragment thereof comprising at least 10 amino acids provided that the fragment comprises the allelic variant at position 233, 256 or 112 of SEQ ID NO: 17.

13. A diagnostic kit comprising an antibody of claim 12.

14. A diagnostic kit comprising the allele specific oligonucleotide probe of claim 7.
